(19) United States
(12) Patent Application Publication
(10) Pub. No.: US 2026/0328715 A1
(43) Pub. Date: Oct. 22, 2026

(54) PAVER PAYLOAD MATERIAL DEPOSIT ESTIMATE

(71) Applicant: Caterpillar Paving Products Inc., Brooklyn Park, MN (US)

(72) Inventors: Matthew Thomas HANSON, St. Louis Park, MN (US); Austin BIES, Saint Louis Park, MN (US); Jacob J. McALPINE, Otsego, MN (US); John Lee MARSOLEK, Watertown, MN (US)

(73) Assignee: Caterpillar Paving Products Inc., Brooklyn Park, MN (US)

(21) Appl. No.: 19/183,734

(22) Filed: Apr. 18, 2025

(51) Int. Cl. G01N 25/18

(52) U.S. Cl. CPC G01N 25/18; E01C 2301/10

(57) ABSTRACT

In some implementations, a paver or remote computer may receive a first temperature indication corresponding to a first temperature of a paving material. The paver or remote computer may receive a second temperature indication corresponding to a second temperature of the paving material. The paver or remote computer may determine a heat transfer value associated with a heat transfer between a screed plate and the paving material. The paver or remote computer may determine or receiving a specific heat capacity associated with the paving material. The paver or remote computer may estimate a mass of the paving material deposited onto a paving surface in accordance with the first temperature indication, the second temperature indication, the heat transfer value, and the specific heat capacity.

### TECHNICAL FIELD

The present disclosure relates generally to a paver and, for example, to estimating an amount of material deposited by a paver.

### BACKGROUND

Asphalt paving is a process where a mixture (also called an “asphalt mixture”) of bitumen (asphalt) and aggregates, such as sand, gravel, or crushed stone, is applied to a surface to form a durable and smooth pavement. The asphalt mixture may be heated, transported, and spread on a prepared surface, such as a roadbed or driveway, and subsequently compacted to achieve a desired thickness and density. A paver is a machine that applies the paving material, such as the asphalt mixture, onto the prepared surface as a compacted mat. The paver may include a hopper for receiving paving material, a distribution system for spreading the material, and a screed for leveling and compacting the material to achieve a desired thickness and surface finish for the compacted mat.

U.S. Pat. No. 10,260,205 (the '205 patent) discloses determining a width and/or a weight and/or a center of gravity of a screed of a road paver by measuring an electrical operating variable of a heating device, such as, in particular, a heating device of the type screed and/or tamper heater, in particular the electrical operating variable of a heating power, a heating resistance or a power consumption of the heating device; and calculating the weight and/or the center of gravity and/or the width of the screed transversely to the operating direction of the screed via the electrical operating variable. The '205 patent further discloses determining a paving volume considering a known paving height. According to the '205 patent, the paving volume can be calculated from the width of the screed if the paving depth is known. The control unit of the paver of the '205 patent is configured to determine the paving volume considering the known paving height and the determined width of the screed transversely to the direction of operation of the screed.

The '205 patent estimates the paving volume based on an estimate of the size of the screed plate and a paving height. The '205 patent assumes that the paving material is uniformly deposited and that the estimate of the size of the screed plate is sufficiently accurate to measure the paving volume. Additionally, the '205 patent fails to consider properties of the paving material when determining the paving volume.

The apparatus, system, and method of the present disclosure solves one or more of the problems set forth above and/or other problems in the art.

### SUMMARY

An apparatus may include one or more memories; and one or more processors, coupled to the one or more memories, configured to: receive a first temperature indication corresponding to a first temperature of a paving material; receive a second temperature indication corresponding to a second temperature of the paving material; determine a heat transfer value associated with a heat transfer between a screed plate and the paving material; determine or receive a specific heat capacity associated with the paving material; estimate a mass of the paving material deposited onto a paving surface in accordance with the first temperature indication, the second temperature indication, the heat transfer value, and the specific heat capacity; and output, to an interface device, a mass indication representing the mass of the paving material deposited onto the paving surface.

A system may include a first temperature sensor configured to output a first temperature indication corresponding to a first temperature of a paving material; a second temperature sensor configured to output a second temperature indication corresponding to a second temperature of the paving material; an interface device; one or more memories; and one or more processors, coupled to the one or more memories, configured to: estimate a mass of the paving material deposited onto a paving surface in accordance with the first temperature indication, the second temperature indication, a heat transfer value, and a specific heat capacity; and output, to the interface device, a mass indication representing the mass of the paving material deposited onto the paving surface.

A method may include receiving a first temperature indication corresponding to a first temperature of a paving material; receiving a second temperature indication corresponding to a second temperature of the paving material; determining a heat transfer value associated with a heat transfer between a screed plate and the paving material; determining or receiving a specific heat capacity associated with the paving material; estimating a mass of the paving material deposited onto a paving surface in accordance with the first temperature indication, the second temperature indication, the heat transfer value, and the specific heat capacity; and outputting, via an interface device, a mass indication representing the mass of the paving material deposited onto the paving surface.

### BRIEF DESCRIPTION OF THE DRAWINGS

FIG. 1 is a diagram associated with an example paver having a screed plate for compacting a boundary area of a paving surface.

FIG. 2 is a diagram of an example system associated with estimating an amount of paving material being deposited onto a paving surface.

FIG. 3 is a diagram of an example system, of a remote computer, associated with estimating an amount of paving material being deposited onto a paving surface by a paver.

FIG. 4 is a flowchart of an example process associated with estimating an amount of paving material being deposited onto a paving surface.

### DETAILED DESCRIPTION

This disclosure relates to estimating an amount of paving material deposited onto a paving surface, which is applicable to one or more construction machines. For example, the construction machine may be a paver.

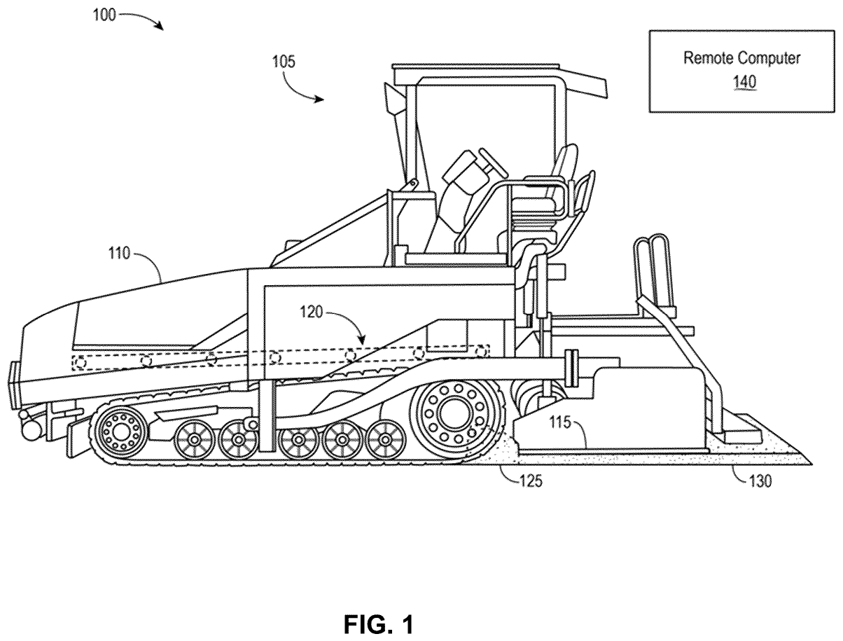

FIG. 1 is a diagram associated with an example paver 100 having a screed plate 115 for compacting a boundary area of a paving surface. As shown in the example of FIG. 1, the paver 100 may include a tractor 105 that includes a front-mounted hopper 110 and a screed plate 115. A conveying system 120 having belts, chains, and/or augers may be configured to transport material (e.g., a hot asphalt mixture 125) from the hopper 110 to the screed plate 115. The screed plate 115 may be configured to level and shape the asphalt mixture into a layer (e.g., a compacted mat 130) having a desired thickness and width. For example, the screed plate 115 may include a screed plate 115 configured to compact the asphalt mixture 125 into the compacted mat 130 parallel to the paving surface. The screed plate 115 may include an electric heater that heats the asphalt mixture 125.

As discussed in greater detail below, the paver 100 may include a system for determining a mass of the asphalt mixture 125 deposited onto the paving surface. The mass M of the asphalt mixture 125 deposited onto the paving surface may correspond to a heat transfer Q between the screed plate 115 and the asphalt mixture 125, a specific heat capacity c of the asphalt mixture 125, and a temperature change ΔT of the asphalt mixture 125 due to the heating of the heating of the asphalt mixture 125 by the screed plate 115. For example, the mass M of the asphalt mixture 125 deposited onto the paving surface may be defined as:

$$M = \frac{Q}{( c \times Δ T )} .$$

As discussed in greater detail below, the paver 100 may include one or more sensors, an on-board computer or controller, or a combination thereof, among other examples, configured to determine the amount of asphalt mixture 125 being deposited onto the paving surface. Alternatively or in addition, the paver 100 may transmit information to a remote computer 140, and the remote computer 140 may be configured to determine the amount of paving material being deposited onto the paving surface. As indicated above, FIG. 1 is provided as an example. Other examples may differ from what is described with regard to FIG. 1.

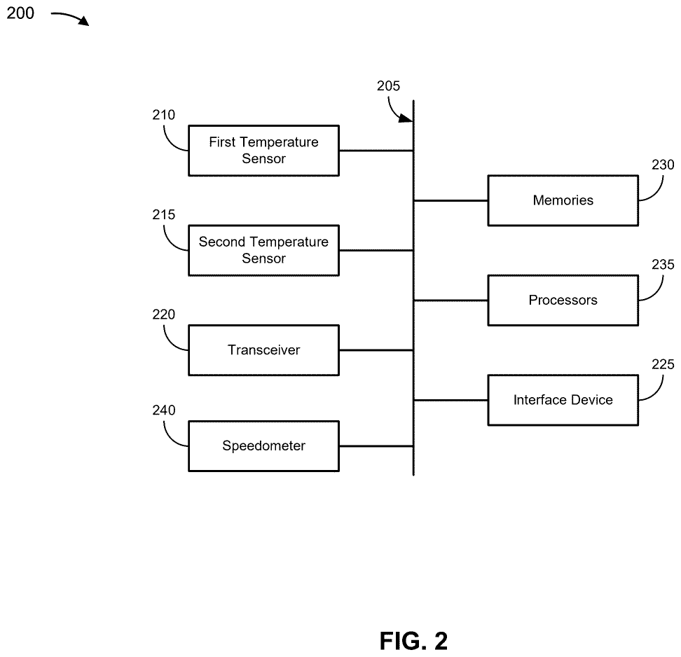

FIG. 2 is a diagram of an example system 200 associated with estimating an amount of paving material being deposited onto a paving surface. As shown in FIG. 2, the paver 100 includes a bus 205, a first temperature sensor 210, a second temperature sensor 215, a transceiver 220, an interface device 225, one or more memories 230, one or more processors 235, and a speedometer 240, among other examples. The components of the system 200 of FIG. 2 may be in communication with one another via the bus 205. The one or more memories 230 and the one or more processors 235 may be part of a controller of the paver 100.

The bus 205 may include one or more components that enable wired and/or wireless communication among the components of the paver 100. The bus 205 couples together two or more components of FIG. 2, such as via operative coupling, communicative coupling, electronic coupling, and/or electric coupling. For example, the bus 205 may include an electrical connection (e.g., a wire, a trace, and/or a lead) and/or a wireless bus.

The first temperature sensor 210 may be configured to measure a temperature of the paving material before the paving material is heated by the screed plate 115. The first temperature sensor 210 may be a thermal imaging sensor or a thermocouple, among other examples. The first temperature sensor 210 may include a sensing element that may generate an electrical signal representative of a temperature value, and may include circuitry for converting the electrical signal into a measurable output. For example, the first temperature sensor 210 may output a first temperature indication representing the temperature of the paving material before the paving material is heated by the screed plate 115. Accordingly, the first temperature sensor 210 may be disposed in the hopper, along the conveying system, or at another location of the paver 100 where the first temperature sensor 210 may measure the temperature of the paving material before the paving material is heated by the screed plate 115.

The second temperature sensor 215 may be configured to measure a temperature of the paving material after the paving material is heated by the screed plate 115. The second temperature sensor 215 may be a thermal imaging sensor or a thermocouple, among other examples. The second temperature sensor 215 may include a sensing element that may generate an electrical signal representative of a temperature value, and may include circuitry for converting the electrical signal into a measurable output. For example, the second temperature sensor 215 may output a second temperature indication representing the temperature of the paving material after the paving material is heated by the screed plate 115. Accordingly, the second temperature sensor 215 may be disposed on or behind (relative to a direction of travel of the paver 100) the screed plate 115, or at another location of the paver 100 where the second temperature sensor 215 may measure the temperature of the paving material after the paving material is heated by the screed plate 115.

The transceiver 220 may include an antenna, circuitry, or other electronic components configured to transmit and receive data signals. The transceiver 220 may include a transmitter module configured to modulate a data signal for transmission and a receiver module configured to demodulate a received signal. The transceiver 220 may include circuitry that may process and convert data signals for communication purposes and may be integrated a communication system of the paver 100. The transceiver 220 may be configured to obtain signals output by other components of the paver 100 shown in FIG. 2 via the bus 205. The transceiver 220 may be configured to wirelessly communicate with the remote computer 140. For example, the transceiver 220 may be configured to receive data from, transmit data to, or a combination thereof, among other examples, the remote computer 140.

The interface device 225 may include a screen, circuitry, or other electronic components configured to receive an input from and/or present information to an operator of the paver 100. For example, the interface device 225 may include a touch screen that may be configured to receive a user input and receive signals for rendering visual representations of data. The interface device 225 may include control circuitry configured to process user inputs and received signals. In some aspects, the interface device 225 may be positioned, on the paver 100, in view of the operator of the paver 100. The interface device 225 may be configured to receive information such as a specific heat capacity, information about the paver 100, information about the screed plate 115, information about the paving material, or a combination thereof, among other examples. Additionally or alternatively, the interface device 225 may be configured to present, to the operator of the paver 100, a visual representation of the amount of paving material being deposited onto the paving surface.

The memory 230 includes volatile and/or nonvolatile memory, such as random access memory (RAM), read only memory (ROM), a hard disk drive, and/or another type of memory (e.g., a flash memory, a magnetic memory, and/or an optical memory). The memory 230 may include internal memory (e.g., RAM, ROM, or a hard disk drive) and/or removable memory (e.g., removable via a universal serial bus connection). In some implementations, the memory 230 is a non-transitory computer-readable medium. The memory 230 stores information, one or more instructions, and/or software (e.g., one or more software applications) related to the operation of the paver 100. For example, the memory 230 may store information associated with the paver 100, the screed plate 115, or the paving material. For example, the memory 230 may store information such as a heat transfer value, a heat transfer factor, a specific heat capacity, or a combination thereof, among other examples, provided via the interface device 225, received from the remote computer 140, or included in firmware, among other examples. The heat transfer value and the heat transfer factor may be associated with a heat transfer between the screed plate 115 and the paving material. The specific heat capacity may be a value corresponding to a specific heat capacity characteristic of the paving material. The heat transfer value, the specific heat capacity, or a combination thereof, among other examples, may be stored in a lookup table in the memory 230. The memory 230 may be configured to store other information, such as the first temperature, the second temperature, a speed of the paver 100, or a combination thereof, among other examples, that may be used to determine the mass of the paving material deposited onto the paving surface. In some implementations, the memory 230 includes one or more memories that are coupled (e.g., communicatively coupled) to one or more processors (e.g., processor 235), such as via the bus 205. Communicative coupling between a processor 235 and a memory 230 enables the processor 235 to read and/or process information stored in the memory 230 and/or to store information in the memory 230.

The processor 235 includes a central processing unit, a graphics processing unit, a microprocessor, a controller, a microcontroller, a digital signal processor, a field-programmable gate array, an application-specific integrated circuit, and/or another type of processing component. The processor 235 may be implemented in hardware, firmware, or a combination of hardware and software. The processor 235 may include one or more processors capable of being programmed to perform one or more operations or processes described elsewhere herein.

The processor 235 may be configured to estimate a mass of the paving material deposited onto a paving surface in accordance with the first temperature indication, the second temperature indication, a heat transfer value, and a specific heat capacity. The processor 235 may be configured to output, to the interface device 225, a mass indication representing the mass of the paving material deposited onto the paving surface. The processor 235 may be configured to receive or access, from the memory 230, the first temperature indication, the second temperature indication, the speed of the paver 100, the heat transfer value, and the specific heat capacity. Rather than access the heat transfer value from the memory 230, the processor 235 may be configured to determine the heat transfer value from an electric current drawn by the screed plate 115. For example, the processor 235 may be configured to receive an electric current measurement associated with the electric current drawn by the screed plate 115. The processor 235 may be configured to determine the heat transfer value by multiplying the electric current measurement by a heat transfer factor. The heat transfer factor may be associated with a property of the screed plate 115, a property of the paving material, or a combination thereof, among other examples. The electric current measurement may be provided to the processor 235 via the memory 230, a heater controller of a heater configured to heat the screed plate 115, an ammeter between the heater controller and the heater of the screed plate 115, or a combination thereof, among other examples. The processor 235 may be configured to determine the mass indication in accordance with a speed of the paver 100. For example, the speed of the paver 100, which may be measured via a speedometer 240, may correspond to an amount of time the paving material is in contact with the screed plate 115. Accordingly, the amount of heat transfer from the screed plate 115 to the paving material may be determined, at least in part, on the speed of the paver 100.

The processor 235 may provide the mass indication to the remote computer 140. The remote computer 140 may be configured to store the mass indication, associate the mass of the paving material being deposited onto the paving surface with a construction site or other indicator, or a combination thereof, among other examples. To provide the mass indication to the remote computer 140, the processor 235 may be configured to cause the transceiver 220 to transmit the mass indication to the remote computer 140.

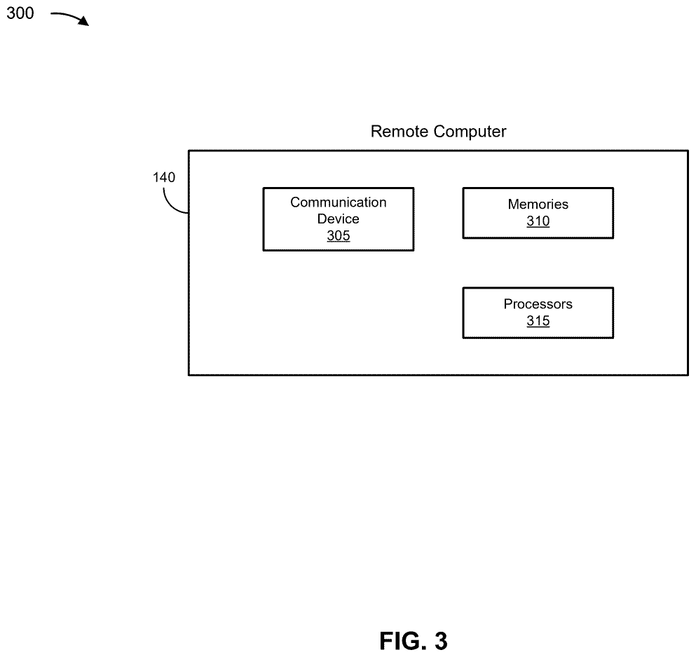

Alternatively, the processor 235 may be configured to receive the mass indication from the remote computer 140. For example, if the processor 235 is not capable of determining the mass indication, the processor 235 may be configured to cause the transceiver 220 to transmit information to the remote computer 140 that may allow the remote computer 140 to calculate the mass of the paving material deposited onto the paving surface. The processor 235 may be configured to receive the mass indication from the remote computer 140 and output the mass indication received from the remote computer 140 to the interface device 225. The processor 235 may be configured to cause the transceiver 220 to transmit the first temperature indication, the second temperature indication, the heat transfer value, the heat transfer factor, the specific heat capacity, the electric current drawn by the screed plate 115, or a combination thereof, among other examples, to the remote computer 140. Additionally, the processor 235 may be configured to cause the transceiver 220 to provide information about the paver 100, the screed plate 115, or a combination thereof, among other examples, to the remote computer 140. As discussed below with respect to FIG. 3, the remote computer 140 may be configured to determine the mass of the paving material deposited onto the paving surface and transmit the mass indication to the paver 100.

In some implementations, the components of FIG. 2, individually or collectively, may perform one or more operations or processes described herein. For example, a non-transitory computer-readable medium (e.g., memory 230) may store a set of instructions (e.g., one or more instructions or code) for execution by the processor 235. The processor 235 may execute the set of instructions to perform one or more operations or processes described herein. In some implementations, execution of the set of instructions, by one or more processors 235, causes the one or more processors 235 and/or other components of FIG. 2 to perform one or more operations or processes described herein. In some implementations, hardwired circuitry is used instead of or in combination with the instructions to perform one or more operations or processes described herein. Additionally, or alternatively, the processor 235 may be configured to perform one or more operations or processes described herein. Thus, implementations described herein are not limited to any specific combination of hardware circuitry and software.

The number and arrangement of components shown in FIG. 2 are provided as an example. The system 200 may include additional components, fewer components, different components, or differently arranged components than those shown in FIG. 2. Additionally, or alternatively, a set of components or systems 200 (e.g., one or more components or systems 200) may perform one or more functions described as being performed by another set of components or systems 200.

FIG. 3 is a diagram of an example system 300, of a remote computer 140, associated with estimating an amount of paving material being deposited onto a paving surface by a paver 100. As shown in FIG. 3, the remote computer 140 includes a communication device 305, one or more memories 310, and one or more processors 315, among other examples.

The communication device 305 may include computers chips, circuitry, or other electronic components configured to transmit and receive data signals. The communication device 305 may include a transmitter module configured to modulate a data signal for transmission and a receiver module configured to demodulate a received signal. The communication device 305 may include circuitry that may process and convert data signals for communication purposes, such as for communication with the paver 100. The communication device 305 may be configured to obtain signals output by other components of the remote computer 140 shown in FIG. 3. The communication device 305 may be configured to wirelessly communicate with the transceiver 220 of the paver 100. For example, the communication device 305 may be configured to receive data from, transmit data to, or a combination thereof, among other examples, the transceiver 220 of the paver 100. Examples of data received at the remote computer 140 via the communication may include information about the paver 100, information about the screed plate 115 of the paver 100, the first temperature indication, the second temperature indication, the heat transfer value, the heat transfer factor, the specific heat capacity, the electric current measurement, or a combination thereof, among other examples.

The memory 310 includes volatile and/or nonvolatile memory, such as RAM, ROM, a hard disk drive, and/or another type of memory (e.g., a flash memory, a magnetic memory, and/or an optical memory). The memory 310 may include internal memory (e.g., RAM, ROM, or a hard disk drive) and/or removable memory (e.g., removable via a universal serial bus connection). In some implementations, the memory 310 is a non-transitory computer-readable medium. The memory 310 stores information, one or more instructions, and/or software (e.g., one or more software applications) related to the operation of the remote computer 140. For example, the memory 310 may store information associated with one or more paving materials, such as one or more heat transfer values, one or more heat transfer factors, one or more specific heat capacities, one or more electric current measurements, or a combination thereof, among other examples. The heat transfer values and the heat transfer factors may be associated with heat transfer between different types of screed plates 135 and different types of paving materials. The specific heat capacities may be values corresponding to specific heat capacity characteristics of different types of paving material. The electric current measurements may correspond to amounts of electric current drawn by one or more screed plates 135. The heat transfer values, the specific heat capacities, the electric current measurements, or a combination thereof, among other examples, may be stored in a lookup table in the memory 310 of the remote computer 140. The memory 310 may be configured to store other information, such as a first temperature, a second temperature, an electric current measurement, or a combination thereof, among other examples, received from the paver 100 and that may be used to determine a mass of the paving material deposited onto the paving surface. The memory 310 may include one or more memories that are coupled (e.g., communicatively coupled) to one or more processors (e.g., processor 315). Communicative coupling between a processor 315 and a memory 310 enables the processor 315 to read and/or process information stored in the memory 310 and/or to store information in the memory 310.

The processor 315 includes a central processing unit, a graphics processing unit, a microprocessor, a controller, a microcontroller, a digital signal processor, a field-programmable gate array, an application-specific integrated circuit, and/or another type of processing component. The processor 315 may be implemented in hardware, firmware, or a combination of hardware and software. The processor 315 may include one or more processors capable of being programmed to perform one or more operations or processes described elsewhere herein.

The processor 315 may be configured to estimate a mass of the paving material deposited onto a paving surface in accordance with the first temperature indication, the second temperature indication, a speed of the paver, a heat transfer value, and a specific heat capacity. The processor 315 may be configured to output, to the communication device 305, a mass indication representing the mass of the paving material deposited onto the paving surface, and the communication device 305 may be configured to transmit the mass indication to the transceiver 220 of the paver 100. To determine the mass of the paving material deposited by a paver 100, the processor 315 may be configured to receive or access, from the memory 310, the first temperature indication, the second temperature indication, the heat transfer value, the specific heat capacity, or a combination thereof, among other examples, for the paver 100. For example, the processor 315 may be configured to query the lookup table in the memory 310 to determine the first temperature indication, the second temperature indication, the heat transfer value, or the specific heat capacity for the paver 100. Alternatively, the processor 315 may be configured to calculate the heat transfer value from the electric current drawn by the screed plate 115 of the paver 100. For example, the processor 315 may be configured to access, from the memory 310, the electric current measurement associated with the electric current drawn by the screed plate 115 of the paver 100. The processor 315 may be configured to access the electric current measurement for the paver 100 by querying the lookup table stored in the memory 310. The processor 315 may be configured to determine the heat transfer value by multiplying the electric current measurement by a heat transfer factor stored in the memory 310 and associated with the paver 100, the screed plate 115, the paving material, or a combination thereof, among other examples. After determining the mass of the paving material deposited onto the paving surface, the processor 315 may output the mass indication to the communication device 305, and the communication device 305 may transmit the mass indication to the paver 100.

In some implementations, the components of FIG. 3, individually or collectively, may perform one or more operations or processes described herein. For example, a non-transitory computer-readable medium (e.g., memory 310) may store a set of instructions (e.g., one or more instructions or code) for execution by the processor 315. The processor 315 may execute the set of instructions to perform one or more operations or processes described herein. In some implementations, execution of the set of instructions, by one or more processors 315, causes the one or more processors 315 and/or other components of FIG. 3 to perform one or more operations or processes described herein. In some implementations, hardwired circuitry is used instead of or in combination with the instructions to perform one or more operations or processes described herein. Additionally, or alternatively, the processor 315 may be configured to perform one or more operations or processes described herein. Thus, implementations described herein are not limited to any specific combination of hardware circuitry and software.

The number and arrangement of components shown in FIG. 3 are provided as an example. The system 300 may include additional components, fewer components, different components, or differently arranged components than those shown in FIG. 3. Additionally, or alternatively, a set of components or systems 300 (e.g., one or more components or systems 300) may perform one or more functions described as being performed by another set of components or systems 300.

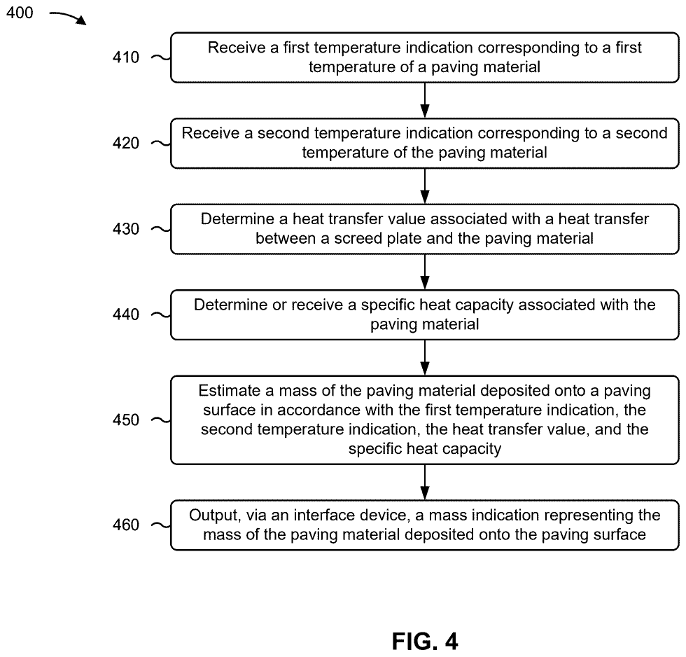

FIG. 4 is a flowchart of an example process 400 associated with estimating an amount of paving material being deposited onto a paving surface. One or more process blocks of FIG. 4 may be performed by a system (e.g., the system 200 or the system 300). Additionally, or alternatively, one or more process blocks of FIG. 4 may be performed by another device or a group of devices separate from or including the system, such as another device or component that is internal or external to the paver 100, the remote computer 140, or a combination thereof, among other examples.

As shown in FIG. 4, process 400 may include receiving a first temperature indication corresponding to a first temperature of a paving material (block 410). For example, the system may receive a first temperature indication corresponding to a first temperature of a paving material, as described above. Receiving the first temperature indication may include receiving the first temperature indication output by a first temperature sensor 210. As discussed above, the first temperature indication may be measured by the first temperature sensor 210 before the paving material is heated by the screed plate 115.

As further shown in FIG. 4, process 400 may include receiving a second temperature indication corresponding to a second temperature of the paving material (block 420). For example, the system may receive a second temperature indication corresponding to a second temperature of the paving material, as described above. Receiving the second temperature indication may include receiving the second temperature indication output by a second temperature sensor 215. The second temperature may be measured by the second temperature sensor 215 after the paving material is heated by the screed plate 115.

As further shown in FIG. 4, process 400 may include determining a heat transfer value associated with a heat transfer between a screed plate 115 and the paving material (block 430). For example, the system may determine a heat transfer value associated with a heat transfer between a screed plate 115 and the paving material, as described above. Determining the heat transfer value may include receiving an electric current measurement associated with an electric current drawn by the screed plate 115 and multiplying the electric current measurement by a heat transfer factor.

As further shown in FIG. 4, process 400 may include determining or receiving a specific heat capacity associated with the paving material (block 440). For example, the system may determine or receive a specific heat capacity associated with the paving material, as described above.

As further shown in FIG. 4, process 400 may include estimating a mass of the paving material deposited onto a paving surface in accordance with the first temperature indication, the second temperature indication, the heat transfer value, and the specific heat capacity (block 450). For example, the system may estimate a mass of the paving material deposited onto a paving surface in accordance with the first temperature indication, the second temperature indication, the heat transfer value, and the specific heat capacity, as described above.

As further shown in FIG. 4, process 400 may include outputting, via an interface device 225, a mass indication representing the mass of the paving material deposited onto the paving surface (block 460). For example, the system may output, via an interface device 225, a mass indication representing the mass of the paving material deposited onto the paving surface, as described above.

The process 400 may include measuring the first temperature indication with a first temperature sensor 210; and outputting, via the first temperature sensor 210, the first temperature indication. Alternatively or in addition, the process 400 may include measuring the second temperature indication with a second temperature sensor 215, and outputting, via the second temperature sensor 215, the second temperature indication. Alternatively or in addition, the process 400 may include transmitting the first temperature and the second temperature to a remote computer 140. Alternatively or in addition, the process 400 may include transmitting one or more of the heat transfer value or a heat transfer factor to the remote computer 140. Alternatively or in addition, the process 400 may include transmitting, to the remote computer 140, an electric current measurement associated with an electric current drawn by the screed plate 115. Alternatively or in addition, the process 400 may include transmitting the mass indication to a remote computer 140.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

### INDUSTRIAL APPLICABILITY

The paver, system, or remote computer, individually or collectively, may provide an operator of the paver with real-time estimates of an amount of paving material (e.g., asphalt mixture) that is being deposited onto a paving surface during a paving operation. The estimate is based on a temperature change of the paving material caused by the screed plate, a heat transfer value, and a specific heat capacity of the paving material. When the operator of the paver is provided with a real-time estimate of the amount of paving material being deposited onto the paving surface, the operator can modify, or continue to perform, the paving operation in real time so that the desired amount of paving material is used. Giving the operator information, such as the amount of paving material being deposited, in real time may prevent waste and avoid costly and time-consuming repairs without compromising the quality of the paved surface.

The foregoing disclosure provides illustration and description, but is not intended to be exhaustive or to limit the implementations to the precise forms disclosed. Modifications and variations may be made in light of the above disclosure or may be acquired from practice of the implementations. Furthermore, any of the implementations described herein may be combined unless the foregoing disclosure expressly provides a reason that one or more implementations cannot be combined. Even though particular combinations of features are recited in the claims and/or disclosed in the specification, these combinations are not intended to limit the disclosure of various implementations. Although each dependent claim listed below may directly depend on only one claim, the disclosure of various implementations includes each dependent claim in combination with every other claim in the claim set.

When “a processor” or “one or more processors” (or another device or component, such as “a controller” or “one or more controllers”) is described or claimed (within a single claim or across multiple claims) as performing multiple operations or being configured to perform multiple operations, this language is intended to broadly cover a variety of processor architectures and environments. For example, unless explicitly claimed otherwise (e.g., via the use of “first processor” and “second processor” or other language that differentiates processors in the claims), this language is intended to cover a single processor performing or being configured to perform all of the operations, a group of processors collectively performing or being configured to perform all of the operations, a first processor performing or being configured to perform a first operation and a second processor performing or being configured to perform a second operation, or any combination of processors performing or being configured to perform the operations. For example, when a claim has the form “one or more processors configured to: perform X; perform Y; and perform Z,” that claim should be interpreted to mean “one or more processors configured to perform X; one or more (possibly different) processors configured to perform Y; and one or more (also possibly different) processors configured to perform Z.”

As used herein, “a,” “an,” and a “set” are intended to include one or more items, and may be used interchangeably with “one or more.” Further, as used herein, the article “the” is intended to include one or more items referenced in connection with the article “the” and may be used interchangeably with “the one or more.” Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”). Further, spatially relative terms, such as “below,” “lower,” “above,” “upper,” and the like, may be used herein for ease of description to describe one element or feature's relationship to another element(s) or feature(s) as illustrated in the figures. The spatially relative terms are intended to encompass different orientations of the apparatus, device, and/or element in use or operation in addition to the orientation depicted in the figures. The apparatus may be otherwise oriented (rotated 90 degrees or at other orientations) and the spatially relative descriptors used herein may likewise be interpreted accordingly.

What is claimed is:

1. An apparatus, comprising:
one or more memories; and
one or more processors, coupled to the one or more memories, configured to:
receive a first temperature indication corresponding to a first temperature of a paving material;
receive a second temperature indication corresponding to a second temperature of the paving material;
determine a heat transfer value associated with a heat transfer between a screed plate and the paving material;
determine or receive a specific heat capacity associated with the paving material;
estimate a mass of the paving material deposited onto a paving surface in accordance with the first temperature indication, the second temperature indication, the heat transfer value, and the specific heat capacity; and
output, to an interface device, a mass indication representing the mass of the paving material deposited onto the paving surface.

2. The apparatus of claim 1, wherein the one or more processors, to determine the heat transfer value, are configured to:
receive an electric current measurement associated with an electric current drawn by the screed plate; and
multiply the electric current measurement by a heat transfer factor.

3. A system, comprising:
a first temperature sensor configured to output a first temperature indication corresponding to a first temperature of a paving material;
a second temperature sensor configured to output a second temperature indication corresponding to a second temperature of the paving material;
an interface device;
one or more memories; and
one or more processors, coupled to the one or more memories, configured to:
estimate a mass of the paving material deposited onto a paving surface in accordance with the first temperature indication, the second temperature indication, a heat transfer value, and a specific heat capacity; and
output, to the interface device, a mass indication representing the mass of the paving material deposited onto the paving surface.

4. The system of claim 3, wherein the one or more processors are configured to determine the heat transfer value in accordance with an electric current measurement and a heat transfer factor,
wherein the electric current measurement is associated with an electric current drawn by a screed plate.

5. The system of claim 3, wherein the first temperature sensor is configured to measure the first temperature before the paving material is heated by a screed plate.

6. The system of claim 5, wherein the second temperature is measured by the second temperature sensor after the paving material is heated by the screed plate.

7. The system of claim 3, further comprising a transceiver configured to transmit the mass indication to a remote computer.

8. A method, comprising:
receiving a first temperature indication corresponding to a first temperature of a paving material;
receiving a second temperature indication corresponding to a second temperature of the paving material;
determining a heat transfer value associated with a heat transfer between a screed plate and the paving material;
determining or receiving a specific heat capacity associated with the paving material;
estimating a mass of the paving material deposited onto a paving surface in accordance with the first temperature indication, the second temperature indication, the heat transfer value, and the specific heat capacity; and
outputting, via an interface device, a mass indication representing the mass of the paving material deposited onto the paving surface.

9. The method of claim 8, wherein determining the heat transfer value includes:
receiving an electric current measurement associated with an electric current drawn by the screed plate; and
multiplying the electric current measurement by a heat transfer factor.

10. The method of claim 8, further comprising measuring the first temperature indication with a first temperature sensor; and
outputting, via the first temperature sensor, the first temperature indication.

11. The method of claim 10, wherein receiving the first temperature indication includes receiving the first temperature indication output by the first temperature sensor.

12. The method of claim 10, wherein the first temperature indication is measured by the first temperature sensor before the paving material is heated by the screed plate.

13. The method of claim 10, further comprising measuring the second temperature indication with a second temperature sensor; and
outputting, via the second temperature sensor, the second temperature indication.

14. The method of claim 13, wherein receiving the second temperature indication includes receiving the second temperature indication output by the second temperature sensor.

15. The method of claim 13, wherein the second temperature is measured by the second temperature sensor after the paving material is heated by the screed plate.

16. The method of claim 8, further comprising transmitting the first temperature and the second temperature to a remote computer.

17. The method of claim 16, further comprising transmitting one or more of the heat transfer value or a heat transfer factor to the remote computer.

18. The method of claim 16, further comprising transmitting, to the remote computer, an electric current measurement associated with an electric current drawn by the screed plate.

19. The method of claim 8, further comprising transmitting the mass indication to a remote computer.
